(19) United States
(12) Patent Application Publication
(10) Pub. No.: US 2021/0267968 A1
(43) Pub. Date: Sep. 2, 2021

(54) Compositions And Methods For Reducing Food Cravings

(71) Applicant: Nalpropion Pharmaceuticals LLC, Morristown, NJ (US)

(72) Inventors: Anthony A. McKinney, San Diego, CA (US); Gary D. Tollefson, Indianapolis, IN (US); Michael Alexander Cowley, Portland, OR (US)

(73) Assignee: Nalpropion Pharmaceuticals LLC, Morristown, NJ (US)

(21) Appl. No.: 17/319,882

(22) Filed: May 13, 2021

Related U.S. Application Data
(63) Continuation of application No. 15/411,585, filed on Jan. 20, 2017, now abandoned.
Application No. 15/411,585 is a continuation of application No. 14/207,077, filed on Mar. 12, 2014, now abandoned.
Application No. 14/207,077 is a continuation of application No. 12/903,131, filed on Oct. 12, 2010, now abandoned.
Application No. 12/903,131 is a continuation of application No. 11/602,813, filed on Nov. 21, 2006, now abandoned.
(60) Provisional application No. 60/739,281, filed on Nov. 23, 2005.

(51) Int. Cl. A61K 31/485; A61K 31/136; A61K 31/137; A61K 31/138; A61K 31/15; A61K 31/19; A61K 31/197; A61K 31/343; A61K 31/351; A61K 31/381; A61K 31/4166; A61K 31/423; A61K 31/451; A61K 31/4525; A61K 31/55; A61K 31/5513; A61K 45/06

(52) U.S. Cl. CPC A61K 31/485; A61K 45/06; A61K 31/137; A61K 31/138; A61K 31/15; A61K 31/19; A61K 31/197; A61K 31/343; A61K 31/351; A61K 31/381; A61K 31/4166; A61K 31/423; A61K 31/451; A61K 31/4525; A61K 31/55; A61K 31/5513; A61K 31/136

(57) ABSTRACT

Disclosed are compositions for reducing food cravings, comprising a first compound and a second compound, where the first compound is an opioid antagonist and the second compound is an α-MSH agonist. Also disclosed are methods of reducing food cravings, comprising identifying an individual in need thereof and treating that individual to antagonize opioid receptor activity and to enhance α-MSH activity.

### RELATED APPLICATION INFORMATION

This application is a continuation of U.S. patent application Ser. No. 14/207,077, filed Mar. 12, 2014, which is a continuation of U.S. patent application Ser. No. 12/903,131, filed Oct. 12, 2010, now abandoned, which is a continuation of U.S. patent application Ser. No. 11/602,813, filed Nov. 21, 2006, now abandoned, which claims priority to and the benefit of U.S. Provisional Patent Application Ser. No. 60/739,281, filed Nov. 23, 2005, each of which is hereby incorporated by reference in its entirety.

### BACKGROUND OF THE INVENTION

### Field of the Invention

The present invention is in the field of pharmaceutical compositions and methods for the reduction of food cravings in individuals.

### Description of the Related Art

Humans crave certain foods at certain times. Certain foods are socially and culturally considered to be “comfort foods,” such as, in the United States, ice cream, chocolate, and meat loaf. Individuals who suffer from temporary sadness or depression crave comfort foods and seek temporary respite from the cause of their unhappiness. It is also well settled that women crave certain foods because of the hormonal changes in their bodies during the normal menstrual cycle or during pregnancy. Some researchers have suggested that normal food cravings may be caused by the lack of certain nutrients in the body. For example, individuals suffering from lack of iron crave crunchy foods, while hypoglycemic individuals crave pasta or bread. While occasional craving is normal in humans, excessive craving can result in poor diet, which can lead to obesity and obesity related complications such as hypertension, non-insulin dependent diabetes mellitus, arteriosclerosis, dyslipidemia, certain forms of cancer, sleep apnea, and osteoarthritis. Excessive food craving can also lead to non-obesity related health problems, such as bulimia.

In certain individuals, a treatment plan that successfully addresses their excessive or abnormal food craving would be highly desirable. Attempts to date have largely been limited to psychological counseling and behavioral changes. These methods are generally not very successful in many individuals, particularly those who have had a long history of craving food and/or abusing food. Therefore, there is a need in the art for a medical treatment to a persistent problem with severe long term implications.

### SUMMARY OF THE INVENTION

In some embodiments, the present invention relates to a method of reducing food cravings comprising identifying a food-craving subject and administering a first compound and a second compound to the subject in an amount that is effective to reduce food craving, wherein the first compound is selected from an opioid antagonist and an anticonvulsant and wherein the second compound is an α-MSH activity enhancer.

The opioid antagonist can be a MOP receptor antagonist. The opioid antagonist can be selected from alvimopan, norbinaltorphimine, nalmefene, naloxone, naltrexone, methylnaltrexone, nalorphine, and pharmaceutically acceptable salts, metabolites or prodrugs thereof. Specifically, the opioid antagonist can be selected from naltrexone and 6-β naltrexol.

The α-MSH activity enhancer can be an α-MSH agonist, triggers the release of α-MSH, and/or increases the activity of neurons that express α-MSH. The α-MSH activity enhancer can be a selective serotonin reuptake inhibitor (SSRI) and/or a specific 5-HT receptor agonist. The SSRI can be selected from fluoxetine, fluvoxamine, sertraline, paroxetine, citalopram, escitalopram, sibutramine, duloxetine, venlafaxine, and pharmaceutically acceptable salts, metabolites or prodrugs thereof. In some embodiments, the α-MSH activity enhancer is bupropion.

The anticonvulsant can be selected from zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide, and pharmaceutically acceptable salts, metabolites or prodrugs thereof. Specifically, the anticonvulsant can be selected from zonisamide, a zonisamide metabolite and a zonisamide prodrug.

In some embodiments, the opioid antagonist is selected from naltrexone, a naltrexone prodrug and a naltrexone metabolite; and wherein the α-MSH activity enhancer is selected from bupropion, a bupropion prodrug and a bupropion metabolite. At least one of the opioid antagonist and the α-MSH activity enhancer can be in a controlled release form, which can be a sustained release form.

In other embodiments, the anticonvulsant is selected from zonisamide, a zonisamide metabolite and a zonisamide prodrug; and wherein the α-MSH activity enhancer is selected from bupropion, a bupropion prodrug and a bupropion metabolite. At least one of the anticonvulsant and the α-MSH activity enhancer can be in a controlled release form, which can be a sustained release form.

The first compound and the second compound can be administered to the subject at about the same time. Alternatively, the first compound can be administered to the subject prior to the second compound, or the first compound and the second compound are combined in a single dosage form.

The first compound and the second compound can be administered to the patient at about the time that the subject experiences the food craving. The first compound and the second compound can be administered to the subject prior to a time period during which the subject typically experiences the food craving. The first compound and the second compound can be administered to the subject in an amount that is effective to synergistically reduce food craving.

In some embodiments, the patient is overweight or obese. In some embodiments, the patient is pregnant.

In some embodiments, the food-craving subject craves a food substance that comprises a carbohydrate. In some embodiments, the food-craving subject craves a food substance that comprises a fat.

In some embodiments, the present invention relates to a package comprising a first compound and a second compound in unit dosage form and written instructions advising the reader to administer the unit dosage form to the intended recipient to alleviate food craving, wherein the first compound is selected from an opioid antagonist and an anticonvulsant and wherein the second compound is an α-MSH activity enhancer.

In some of these embodiments, the opioid antagonist is selected from naltrexone, a naltrexone prodrug and a naltrexone metabolite and the α-MSH activity enhancer is selected from bupropion, a bupropion prodrug and a bupropion metabolite. At least one of the opioid antagonist and the α-MSH activity enhancer can be in a controlled release form, which can be a sustained release form.

In other of these embodiments, the anticonvulsant is selected from zonisamide, a zonisamide prodrug and a zonisamide metabolite and the α-MSH activity enhancer is selected from bupropion, a bupropion prodrug and a bupropion metabolite. At least one of the anticonvulsant and the α-MSH activity enhancer can be in a controlled release form, which can be a sustained release form.

### BRIEF DESCRIPTION OF THE DRAWINGS

FIG. 1. Percentage change in weight corresponding to percentage change in the Food Craving Inventory (FCI) total score corresponding to double placebo treatment (open symbols) and to treatment consisting of 400 mg/day bupropion and 16 mg/day naltrexone (filled symbols). Each symbol corresponds to data from one subject. FCI total scores and the subjects' weights were assessed before treatment and after 24 weeks of treatment to determine the reported percentage change.

FIG. 2. Percentage change in weight corresponding to percentage change in the Food Craving Inventory (FCI) total score corresponding to double placebo treatment (open symbols) and to treatment consisting of 400 mg/day bupropion and 48 mg/day naltrexone (filled symbols). Each symbol corresponds to data from one subject. FCI total scores and the subjects' weights were assessed before treatment and after 24 weeks of treatment to determine the reported percentage change.

### DETAILED DESCRIPTION OF THE PREFERRED EMBODIMENTS

Arcuate nucleus neurons are known to be responsive to a wide array of hormones and nutrients, including leptin, insulin, gonadal steroids, and glucose. In addition to potential transport mechanisms, peripheral substances may access these neurons via arcuate cell bodies in and projections to the median eminence, a region considered to be a circumventricular organ, which lacks a blood-brain barrier. Cone et al., “The arcuate nucleus as a conduit for diverse signals relevant to energy homeostasis,” Int'l Journal of Obesity (2001) 25, Suppl 5, S63-S67.

Administration of exogenous leptin activates a number of different neurons in hypothalamic and brainstem cell groups that bear the leptin receptor. Leptin-responsive neurons in the arcuate nucleus include both those containing neuropeptide Y (NPY) and agouti-related peptide (AgRP) in the medial part of the nucleus and those containing both pro-opiomelanocortin (POMC) and its derivatives, including α-melanocyte stimulating hormone (α-MSH), as well as cocaine and amphetamine-related transcript (CART). Saper et al., “The need to feed: Homeostatic and hedonic control of eating,” Neuron, 36:199-211 (2002).

The leptin-responsive POMC neurons in the arcuate nucleus are thought to cause anorexia and weight reduction by means of the action of α-MSH on melanocortin 3 and/or 4 receptors (MC3-R, MC4-R). The highest MC3-R expression level is in the hypothalamus and limbic system, whereas MC4-R mRNA is expressed in virtually all major brain regions. Some of the metabolic effects resulting from stimulation of MC4-R are decreased food intake and an increase in energy expenditure through stimulation of thyrotropin-releasing hormone and activation of the sympathetic nervous system. Targeted deletion of the MC4-R gene produces obesity, hyperphagia, hyperinsulinemia, and reduced energy expenditure. Targeted deletion of MC3-R results in increased adiposity due to decreased energy expenditure. Korner et al., “The emerging science of body weight regulation and its impact on obesity treatment,” J. Clin. Invest. 111(5):565-570 (2003). Thus, increased concentrations of α-MSH in the central nervous system (CNS) increase its action on MC3-R and/or MC4-R and result in a suppressed appetite.

POMC neurons also release β-endorphin when they release α-MSH. β-endorphin is an endogenous agonist of the μ-opioid receptors (MOP-R), found on the POMC neurons. Stimulation of MOP-R decreases the release of α-MSH. This is a biofeedback mechanism that under normal physiological conditions controls the concentration of α-MSH in the CNS. Thus, blocking MOP-R by opioid antagonists will break the feedback mechanism, which results in continued secretion of α-MSH and an increase in its concentration in the CNS.

A second population of neurons in the arcuate nucleus tonically inhibits the POMC neurons. These POMC-inhibiting neurons secrete NPY, the neurotransmitter γ-aminobutyric acid (GABA), and AgRP. NPY and GABA inhibit POMC neurons, via NPY Y1 receptors and GABA receptors, respectively. Thus, within the arcuate nucleus NPY and GABA inhibit the release of α-MSH, and therefore are stimulators of feeding. It is known that leptin inhibits the release of GABA from NPY terminals synapsing onto POMC neurons, whereas ghrelin, an orexigenic peptide, stimulates the ghrelin receptors on NPY neurons and increase the secretion of NPY and GABA onto the POMC cells, which in turn inhibits the release of α-MSH.

AgRP stimulates food intake in the rat through antagonism of the interaction of α-MSH at MC4-R. Expression of the AgRP gene is suppressed by leptin.

Serotonin, also known as 5-hydroxytryptamine or 5-HT, activates the POMC neurons to secrete α-MSH. However, serotonin is taken up and removed from action by specific transporters so that a single serotonin molecule has short term effects. It is known that selective serotonin re-uptake inhibitors (SSRIs) prevent the uptake of serotonin and increase its concentrations in the CNS. Thus, SSRIs also increase the secretion of α-MSH and its concentrations in the CNS.

Dopamine also increases the activity of POMC neurons to secrete α-MSH. Like serotonin, dopamine is also taken up and removed from action so that a single dopamine molecule has short term effect. Dopamine re-uptake inhibitors, which prevent or reduce the uptake of dopamine, can also increase the secretion of α-MSH and its concentrations in the CNS.

Therefore, increased secretion of α-MSH through various mechanisms, such as serotonin re-uptake inhibition, are among the strategies that the methods and pharmaceutical compositions of embodiments of the present invention pursue in order to reduce food cravings. A preferred embodiment provides a multi-faceted combination therapy approach to the problem of reducing food cravings. It addresses not just single molecules, messengers, or receptors, but instead acts on multiple points in the feeding and satiety pathway. Aspects of a preferred embodiment are directed to increasing the concentrations of α-MSH in the CNS by stimulating the release of α-MSH, suppressing its metabolism, reducing the antagonism of its interaction at MC3/4-R, and suppressing any feedback mechanisms that slow or stop its release. Aspects of a preferred embodiment include pharmaceutical compositions whose components achieve one or more of these functions.

Thus, in a first aspect, the present invention relates to a method of reducing cravings for one or more craved substances, comprising identifying an individual in need thereof and treating that individual to antagonize opioid receptor activity and to enhance α-MSH activity. In some embodiments, the opioid receptor activity is antagonized by a first compound, where the first compound is an opioid antagonist and the α-MSH activity is enhanced by a second compound, where the second compound is a melanocortin 3 receptor (MC3-R) or a melanocortin 4 receptor (MC4-R) agonist In preferred embodiments, the present invention relates to a method of reducing food cravings, comprising identifying an individual in need thereof and treating that individual to antagonize opioid receptor activity and to enhance α-MSH activity. In another aspect, the present invention relates to a method of reducing cravings for one or more craved substances, comprising identifying an individual in need thereof and treating that individual with an anticonvulsant and to enhance α-MSH activity.

### Definitions

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The terms “serotonin 1B receptor,” “serotonin 2C receptor,” “5-HT1b receptor,” and “5-HT2c receptor” refer to receptors found more commonly in rodents. It is understood by those of skill in the art that other mammals have serotonin receptors on various neurons that are analogous in function and form to these receptors. Agonists or antagonists at these non-rodent, preferably human, serotonin receptors are within the scope of the present invention.

A used herein, a “food craving” subject is a person who experiences a strong, sometimes intense or overwhelming desire for a particular food substance or type of craved food substance. Food cravings can be measured by various methods known to those skilled in the art. In preferred embodiments, food cravings are measured by the Food Craving Inventory (FCI). The FCI is a reliable and valid self-report measure of general and specific food cravings (see White et al., Obesity Research, 10(2), 107-114, 2002). In other embodiments, other methods can be used to measure food cravings, such as the Yale Brown Obsessive Compulsive Scale and variants thereof.

A “craved food substance” can be any food or drink-related substance that is the subject of a food craving. Examples of craved food substances include sweetened-food substances such as baked goods (e.g., cookies, brownies, pies, cakes, and the like); candy-based substances (e.g., hard candy, soft candy, chewable candy, gums, and the like), dairy-based products such as ice cream, yogurts, cheeses, chocolate milk, sweetened milk, and the like; and salty snack foods such as potato chips, pretzels, popcorn, and the like. As used herein, craved food substances includes liquid or beverage-based substances such as fruit and/or juice-based beverages, chocolate-based beverages (e.g., hot chocolate), non-alcoholic beverages, carbonated beverages, sweetened beverages, non-sweetened beverages, and the like. To the extent that the food craving is directed to the food aspects of an alcoholic beverage and not to the alcoholic aspects, an alcoholic beverage can be a craved food substance. As used herein, a food craving is not a craving for alcohol. In some embodiments, the craved substance can include a large variety of foods, such that the subject may indicate that he craves food in general. In other embodiments, the craved substance can be a specific food category, such as sweets, carbohydrates, or fats. In some preferred embodiments, the craved substance is carbohydrates. The “craved food substance” can be in any suitable delivery formulation, such as the examples provided above. The craved food substance can be a variety of foods or beverages, a category of foods or beverages, or a specific food or beverage.

### Opioid Antagonists

A variety of opioid antagonists and combinations thereof are suitable for use in the methods and compositions described herein. In some embodiments, opioid receptor activity is antagonized by administering an opioid receptor antagonist. In certain embodiments the opioid antagonist antagonizes a μ-opioid receptor (MOP-R) in a mammal. The opioid antagonist may be a MOP receptor antagonist. In some embodiments, the opioid antagonist is selected from alvimopan, norbinaltorphimine, nalmefene, naloxone, naltrexone, methylnaltrexone, nalorphine, and pharmaceutically acceptable salts, metabolites or prodrugs thereof. In some embodiments, the opioid antagonist is naltrexone, a naltrexol metabolite (e.g., 6-β naltrexol), a prodrug of naltrexone or a prodrug of a naltrexone metabolite.

In other embodiments, the opioid antagonist is a partial opioid agonist. Compounds of this class have some agonist activity at opioid receptors. However, because they are weak agonists, they function as de-facto antagonists. Examples of partial opioid agonists include pentacozine, buprenorphine, nalorphine, propiram, and lofexidine. Routine experimentation, informed by the guidance provided herein, may be used to identify an opioid antagonist suitable for use in combination with a particular α-MSH activity enhancer in the methods and compositions described herein.

### Enhanced α-MSH Activity

A variety of compounds and combinations thereof are suitable for use in enhancing the activity of α-MSH in the methods and compositions described herein. In certain embodiments opioid receptor activity is antagonized by a first compound and the α-MSH activity is enhanced by a second compound, where the second compound causes increased agonism of a melanocortin 3 receptor (MC3-R) or a melanocortin 4 receptor (MC4-R) compared to normal physiological conditions and can trigger the release of α-MSH or increases the activity of neurons that express α-MSH. In some embodiments, the second compound causes increased activity of the POMC neurons, leading to greater agonism at MC3-R and/or MC4-R. Compounds that enhance α-MSH activity may be referred to herein as α-MSH activity enhancers.

In certain embodiments, the α-MSH activity enhancer triggers the release of α-MSH. The α-MSH activity enhancer may increase the extracellular serotonin concentrations in the hypothalamus. In some embodiments, the α-MSH activity enhancer is selected from the group consisting of a selective serotonin reuptake inhibitor (S SRI), a serotonin 2C agonist, and a serotonin 1B agonist. In further embodiments, the α-MSH activity enhancer is selected from the group consisting of fluoxetine, fluvoxamine, sertraline, paroxetine, citalopram, escitalopram, sibutramine, duloxetine, and venlafaxine, and pharmaceutically acceptable salts, metabolites or prodrugs thereof.

In certain embodiments, the α-MSH activity enhancer suppresses the expression of the AgRP gene or the production or release of agouti-related protein (AgRP). In some of these embodiments, the α-MSH activity enhancer suppresses the activity of neurons that express AgRP.

In other embodiments, the α-MSH activity enhancer suppresses the expression of the NPY gene or the production or release of neuropeptide Y (NPY). In some of these embodiments, the α-MSH activity enhancer suppresses the activity of neurons that express NPY. In further embodiments, the α-MSH activity enhancer is selected from the group consisting of NPY antagonists, ghrelin antagonists, and leptin. In certain other embodiments, the second compound agonizes NPY Y2 receptor.

Other embodiments of the present invention include those in which the α-MSH activity enhancer is selected from the group consisting of a γ-amino butyric acid (GABA) inhibitor, a GABA receptor antagonist, and a GABA channel antagonist. By “GABA inhibitor” it is meant a compound that reduces the production of GABA in the cells, reduces the release of GABA from the cells, or reduces the activity of GABA on its receptors, either by preventing the binding of GABA to GABA receptors or by minimizing the effect of such binding. The GABA inhibitor may be a 5-HT1b agonist or another agent that inhibits the activity of NPY/AgRP/GABA neurons. In addition, the GABA inhibitor may suppress the production or release of the AgRP gene, or the GABA inhibitor may suppress the production or release of AgRP. It is, however, understood that a 5-HT1b agonist may inhibit the NPY/AgRP/GABA neuron (and therefore activate POMC neurons) without acting as an inhibitor of the GABA pathway.

In certain other embodiments the GABA inhibitor increases the expression of the POMC gene. In some of these embodiments, the GABA inhibitor increases the production or release of pro-opiomelanocortin (POMC) protein. In certain other of these embodiments, the GABA inhibitor increases the activity on POMC expressing neurons. In some embodiments, the GABA inhibitor is topiramate, 1-(2-(((diphenylmethylene)amino)oxy)ethyl)-1,2,5,6-tetrahydro-3-pyridinecarboxylic acid hydrochloride (NNC-711), or vigabatrin.

In other embodiments the α-MSH activity enhancer is a dopamine reuptake inhibitor. Phentermine is an example of a dopamine reuptake inhibitor. In certain other embodiments, the α-MSH activity enhancer is a norepinephrine reuptake inhibitor. Examples of norepinephrine reuptake inhibitors include bupropion, thionisoxetine, and reboxetine. Other embodiments include those in which the α-MSH activity enhancer is a dopamine agonist. Some dopamine agonists that are available on the market include cabergoline, amantadine, lisuride, pergolide, ropinirole, pramipexole, and bromocriptine. In further embodiments, the second compound is a norepinephrine releaser, for example diethylpropion, or a mixed dopamine/norepinephrine reuptake inhibitor, for example, atomoxatine.

In some embodiments, the α-MSH activity enhancer is bupropion. In other embodiments, the second compound is a metabolite of bupropion. The metabolites of bupropion suitable for inclusion in the methods and compositions disclosed herein include the erythro- and threo-amino alcohols of bupropion, the erythro-amino diol of bupropion, and morpholinol metabolites of bupropion. In some embodiments, the metabolite of bupropion is (±)-(2R*,3R*)-2-(3-chlorophenyl)-3,5,5-trimethyl-2-morpholinol. In some embodiments the metabolite is (−)-(2R*,3R*)-2-(3-chlorophenyl)-3,5,5-trimethyl-2-morpholinol, while in other embodiments, the metabolite is (+)-(2S,3S)-2-(3-chlorophenyl)-3,5,5-trimethyl-2-morpholinol. Preferably, the metabolite of bupropion is (+)-(2S,3S)-2-(3-chlorophenyl)-3,5,5-trimethyl-2-morpholinol, which is known by its common name of radafaxine, which is described in U.S. Pat. No. 6,274,579, issued on Aug. 14, 2001 to Morgan et al., which is hereby incorporated by reference herein in its entirety, including any drawings.

In certain other embodiments, the α-MSH activity enhancer is a 5-HT1b agonist, such as sumatriptan, almotriptan, naratriptan, frovatriptan, rizatriptan, zomitriptan, and elitriptan.

In other embodiments, the α-MSH activity enhancer is used in combination with an anticonvulsant. The anticonvulsant may be selected from the group consisting of zonisamide, topiramate, nembutal, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide.

In certain embodiments, the α-MSH activity enhancer may be a combination of two or more compounds. For example, the second compound may be a combination of a dopamine reuptake inhibitor and a norepinephrine reuptake inhibitor, e.g. bupropion and mazindol. Alternatively, the second compound may be a combination of a SSRI and a norepinephrine reuptake inhibitor, such as sibutramine, venlafaxine, and duloxetine.

In certain embodiments, the second compound is an activator of the POMC neurons. Examples of POMC activators include Ptx1 and interleukin 1 beta, (IL-1β).

In some of the embodiments set forth above, α-MSH activity is enhanced by administering a compound, where the compound triggers release of α-MSH or increases the activity of neurons that express α-MSH. In some embodiments, the compound is a selective serotonin reuptake inhibitor (SSRI) or a specific 5-HT receptor agonist. Examples of SSRIs that can be used in the present invention include fluoxetine, fluvoxamine, sertraline, paroxetine, citalopram, escitalopram, sibutramine, duloxetine, and venlafaxine, and pharmaceutically acceptable salts or prodrugs thereof. Routine experimentation, informed by the guidance provided herein, may be used to identify an α-MSH activity enhancer suitable for use in combination with a particular opioid antagonist in the methods and compositions described herein.

### Compound Combinations

In some embodiments, the following combinations of compounds are administered or comprised in a composition:

a SSRI in combination with a dopamine reuptake inhibitor, a dopamine/norepinephrine reuptake inhibitor, a norepinephrine reuptake inhibitor, an opioid antagonist, a partial opioid agonist, GABA inhibitor, a peripherally acting weight loss agent such as metformin, or a peptide, such as PYY, PYY₃₋₃₆, or leptin;

Serotonin in combination with a dopamine reuptake inhibitor, a dopamine/norepinephrine reuptake inhibitor, an opioid antagonist, a partial opioid agonist, or a GABA inhibitor;

a dopamine reuptake inhibitor in combination with a norepinephrine reuptake inhibitor, a norepinephrine releaser, a norepinephrine agonist, an opioid antagonist, a partial opioid agonist, a GABA inhibitor, an adenosine compound, a cholinergic receptor antagonist, or a peptide, such as PYY, PYY₃₋₃₆, or leptin;

a dopamine/norepinephrine reuptake inhibitor in combination with an opioid antagonist, a partial opioid agonist, a GABA inhibitor, or a peripherally acting weight loss agent such as metformin;

a dopamine agonist in combination with an opioid antagonist, a partial opioid agonist, a GABA inhibitor, or a peptide, such as PYY, PYY₃₋₃₆, or leptin.

Examples of norepinephrine agonists include phendimetrazine and benzphetamine. Examples of adenosine compounds include all xanthine derivatives, such as adenosine, caffeine, theophylline, theobromine, and aminophylline. An example of a cholinergic receptor antagonist is nicotine.

In some embodiments, the following combinations of compounds are administered or comprised in a composition:

an opioid antagonist and an α-MSH activity enhancer, wherein the opioid antagonist is selected from naltrexone, a naltrexone prodrug and a naltrexone metabolite; and wherein the α-MSH activity enhancer is selected from bupropion, a bupropion prodrug and a bupropion metabolite;

an anticonvulsant and an α-MSH activity enhancer, wherein the anticonvulsant is selected from zonisamide, a zonisamide metabolite and a zonisamide prodrug; and wherein the α-MSH activity enhancer is selected from bupropion, a bupropion prodrug and a bupropion metabolite;

an opioid antagonist, an anticonvulsant and an α-MSH activity enhancer, wherein the opioid antagonist is selected from naltrexone, a naltrexone prodrug and a naltrexone metabolite; the anticonvulsant is selected from zonisamide, a zonisamide metabolite and a zonisamide prodrug; and wherein the α-MSH activity enhancer is selected from bupropion, a bupropion prodrug and a bupropion metabolite.

### Methods

In some embodiments, the present invention relates to a method of reducing cravings (e.g. food cravings) in an individual comprising identifying an individual in need thereof and treating that individual with an anticonvulsant and/or to antagonize opioid receptor activity, and to enhance α-MSH activity. In some embodiments, the treating step of the above method comprises administering to the individual a first compound and a second compound, where the first compound is an opioid antagonist and/or an anticonvulsant, and the second compound enhances α-MSH activity. In some embodiments, the individual is treated with naltrexone and bupropion; in other embodiments the individual is treated with zonisamide and bupropion.

Some embodiments of the invention include methods of treating an overweight or an obese patient, comprising identifying an overweight or an obese patient and administering one or more compositions described herein to the patient. Such administration can reduce food intake overall or intake of specific food resulting in the patient losing weight. One or more of the compositions described herein can also be used to suppress the appetite of a patient.

Other embodiments of the invention include methods of treating a patient suffering or at risk of suffering from a condition in which it is undesirable to eat certain foods, such as Type-2 diabetes. These methods can comprise identifying a patient suffering or at risk of suffering from such a disease and administering one or more compositions described herein to the patient. Such administration can inhibit the food cravings and thereby inhibit the progression of the disease.

### Patient Identification

Compositions described herein can be administered to a subject craving a craved substance. The patient subject be selected from the group consisting of mice; rats; rabbits; guinea pigs; dogs; cats; sheep; goats; cows; primates, such as monkeys, chimpanzees, and apes; and humans. In certain embodiments, the patient is overweight, which is characterized by a body mass index (BMI) greater than 25. In other embodiments, the patient is obese, which is characterized by a BMI greater than 30. In still other embodiments, the individual has a BMI greater than 40. However, in some embodiments, the subject may have a BMI less than 25. In these embodiments, it may be beneficial for health or cosmetic purposes to reduce food cravings. For example, the subject may be suffering from or be at risk of bulimia.

In some embodiments, the patient is suffering from or at risk of suffering from a condition in which it is undesirable to eat certain foods, such as Type-2 diabetes. In some of these embodiments, the condition may be related to the patient being overweight. The condition may also be inhibited by weight loss. In some embodiments, the patient is being administered a different medication which causes an increase in food cravings. In other embodiments, the patient is pregnant.

As used herein, the phrase “a food-craving subject” includes subjects who are currently experiencing food cravings, subjects who have previously experienced food cravings, and subjects who are likely to experience food cravings.

In some embodiments, the patient is also experiencing cravings for a non-food substance, such as alcohol, pain relievers, tranquilizers, depressants, sleep aids, tobacco substances, cocaine, or marijuana.

### Administration

In some embodiments, the subjects are administered a composition comprising a first compound, which is an anticonvulsant and/or an opioid antagonist, and a second compound, which enhances α-MSH activity. In some of these embodiments, the first compound and the second compound are administered nearly simultaneously. These embodiments include those in which the two compounds are in the same administrable composition, i.e., a single unit dosage form such as a single tablet, pill, or capsule, or a single solution for intravenous injection, or a single drinkable solution, or a single dragee formulation or patch, contains both compounds. The first compound and the second compound can be covalently linked to each other such that they form a single chemical entity. The single chemical entity is then digested and is metabolized into two separate physiologically active chemical entities, one of which is the first compound and the other one is the second compound.

The embodiments also include those in which each compound is in a separate administrable composition or unit dosage form, but the patient is directed to take the separate compositions nearly simultaneously, e.g., one pill is taken right after the other or one injection of one compound is made right after the injection of another compound, etc. In some embodiments, a patient is infused with an intravenous formulation of one compound prior to the infusion of an intravenous formulation of the other compound. In these embodiments, the infusion may take some time, such as a few minutes, a half hour, or an hour, or longer. If the two intravenous infusions are done one right after the other, such administration is considered to be nearly simultaneously within the scope of the present disclosure, even though there was a lapse of some time between the start of one infusion and the start of the next infusion. In other embodiments the first compound is administered prior to the second compound. In yet other embodiments, the first compound is administered subsequent to the second compound.

In other embodiments the administering step comprises administering one of the first compound and the second compound first and then administering the other one of the first compound and the second compound. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally. In further embodiments, the patient may receive both compounds on a routine or continuous basis, such a continuous infusion of the compound through an IV line.

In certain embodiments disclosed herein, an individual is given a pharmaceutical composition comprising a combination of two or more compounds to reduce cravings. In some of these embodiments, each compound is a separate chemical entity. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

Thus, in another aspect, the present invention relates to synthetic routes to novel molecules in which an opioid antagonist is linked by a flexible linker to a selective serotonin reuptake inhibitor (S SRI).

### Route of Administration and Formulation

The exact formulation and route of administration for the pharmaceutical compositions described herein can be chosen by the individual physician in view of the patient's condition. (See e.g., Fingl et al. 1975, in “The Pharmacological Basis of Therapeutics”, Ch. 1 p. 1). Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipients with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in a mixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

Controlled release forms of the compositions described herein are specifically contemplated, including sustained release formulations. Methods for formulating controlled release forms are known to those skilled in the art and may be applied to make controlled release compositions using routine experimentation informed by the guidance provided herein.

The compositions described herein can be included in a food or beverage product. In some preferred embodiments, the compositions can be included in a food or beverage product similar to the craved substance. The food or beverage product can be a beverage, a soup, a solid, a semi-solid, or a frozen confection. The beverage can be a still beverage or a carbonated beverage, and moreover, can be a suspension, for example, a shake, frappe, or float. Carbonated beverages are preferably made without phosphoric acid, to permit a higher pH. Carbonated beverages as diluents are preferably used with buffer formulations of the composition, such that the final pH is greater than about 6. Both carbonated and non-carbonated beverages can be “diet” beverages made with low calorie or no-calorie sweeteners, including saccharine, aspartame, dihydrochalcones, monellin, steviosides, glycyrrhizin, sorbitol, mannitol, maltitol, and others. The beverage can be an infusion or extract, including a tea or a coffee. The solid can be a bar, much like an energy bar or a candy bar; a chip, like a potato or corn chip in shape or texture; a baked good; a non-baked extruded food product; a puffed snack; a cracker; a cookie; in which the solid can be with or without embedded flavor nuggets such as nuts, fruits, or chocolate chips. The semi-solid snack can be a custard, a dessert pudding, a thick cream, a mousse, a parfait, a yogurt, a jelly, a sweetened gelatin, and similar snacks. The frozen confection can be an “ice cream”, an “ice milk”, a sherbet, a flavored ice, and similar snacks, and can, optionally, include a wafer or cone, a stick, cup, or flavor nuggets such as nuts and candy sprinkles (a.k.a. “jimmies”). The frozen confection can be formed into any of a variety of attractive shapes including cones, cups, bars, and sandwiches. The compounds can also be in a powder form. Preferably the powder is free-flowing and readily mixable with water or other fluid. The powder can be mixed with a variety of fluids. Thus, for example, the powder form of the invention can be mixed with water, soda, diet soda, tea, coffee, fruit juice, diet fruit juice, flavored diet beverages, and the like. Preferably, the powder form of the invention is mixed with water or other fluid before drinking. In some embodiments, the compounds can be delivered in the form of a toothpaste.

For buccal administration, the compositions may take the form of tablets or lozenges formulated in conventional manner.

For administration by inhalation, the compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Pharmaceutical Carriers

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few minutes up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

### Dosages

Pharmaceutical compositions suitable for use in the present invention include compositions where the active ingredients are contained in an amount effective to achieve its intended purpose. In some embodiments, a therapeutically effective amount means an amount of compound effective to reduce, and in preferred embodiments to substantially reduce, cravings of the subject being treated. In other embodiments, a therapeutically effective amount means an amount of compound effective to reduce, and in preferred embodiments to substantially reduce, the weight of the subject being treated. In still other embodiments, a therapeutically effective amount means an amount of compound effective to prevent, alleviate or ameliorate the treated subject's symptoms of a disease in which it is undesirable to eat specific foods. Determination of a therapeutically effective amount is well within the capability of those skilled in the art, especially in light of the detailed disclosure provided herein. An amount of a compound that is effective to reduce food craving is an amount that reduces food craving as measured by any one of the various methods known to those skilled in the art for assessing food craving. Preferred methods include the Food Craving Inventory (FCI) methods. In some embodiments, the methods include the Yale Brown Obsessive Compulsive Scale (YBOCS) and variants thereof to assess food cravings.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The composition can be administered in a controlled-release dosage form. The composition can be administered to the patient before, during, or after a specific meal or before, during, or after every meal. The composition can be administered to the patient when the patient experiences a food craving, or at various periods of time before the patient typically experiences the food craving. The composition can be administered before the patient goes to sleep or in the morning.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

An embodiment provides a package comprising a first compound and a second compound in unit dosage form as described herein, along with instructions advising the reader to administer the unit dosage form to the intended recipient to alleviate food craving.

### Food Cravings

Some of the methods and compositions provided herein can reduce cravings of craved substances. Several techniques can be used to determine whether cravings were reduced by one of the disclosed methods and/or compositions. In one embodiment, the patient can indicate whether the method and/or composition reduced the cravings. In another embodiment, the consumption of the craved substances can be measured to determine whether the method and/or composition reduced the cravings. In preferred embodiments, the efficacy of the method and/or composition of reducing food cravings can be measured by the Food Craving Inventory (FCI). The FCI is a reliable and valid self-report measure of general and specific food cravings (White et al., Obesity Research, 10(2), 107-114, 2002). The FCI measures specific food cravings using two subscales: subjective cravings and consumption of particular foods. In other embodiments, the Yale Brown Obsessive Compulsive Scale modified for food craving (Food Craving YBOCS) can measure food cravings. In other methods, other methods can be used to assess food cravings, such as the Yale Brown Obsessive Compulsive Scale modified for food craving (Food Craving YBOCS). The YBOCS is a well-accepted method for quantifying the severity of symptoms of obsessive-compulsive disorder which is not specific to the type of symptoms experienced, Goodman et al., Arch. Gen. Psychiatry (1989) 46:1006-11. The modified Food Craving YBOCS quantifies the severity of symptoms associated with food cravings. The Food Craving YBOCS utilizes subjective measurements regarding the extent to which food cravings interfere with an individual's daily activities. The FCI, YBOCS, and/or another food-craving assessment method can be administered before the method is applied and/or the composition is administered and after the method has been applied and/or the composition has been administered for some time.

It will be understood by those of skill in the art that numerous and various modifications can be made without departing from the spirit of the present invention. Therefore, it should be clearly understood that the forms of the present invention are illustrative only and are not intended to limit the scope of the present invention.

### Some Embodiments of the Invention

In the first embodiment, the invention relates to a method of reducing cravings, comprising identifying an individual in need thereof and treating that individual to antagonize opioid receptor activity and to enhance α-MSH activity.

In the second embodiment, the invention relates to the method of the first embodiment, wherein said individual has a body mass index greater than 25.

In the third embodiment, the invention relates to the method of the first embodiment, wherein opioid receptor activity is antagonized by administering an opioid receptor antagonist.

In the fourth embodiment, the invention relates to the method of the third embodiment, wherein the opioid receptor antagonist is a MOP receptor antagonist.

In the fifth embodiment, the invention relates to the method of the first embodiment, wherein the opioid receptor antagonist is selected from alvimopan, norbinaltorphimine, nalmefene, naloxone, naltrexone, methylnaltrexone, and nalorphine, and pharmaceutically acceptable salts or prodrugs thereof.

In the sixth embodiment, the invention relates to the method of the third embodiment, wherein said opioid receptor antagonist is a partial opioid agonist.

In the seventh embodiment, the invention relates to the method of the sixth embodiment, wherein said partial opioid agonist is selected from the group consisting of pentacozine, buprenorphine, nalorphine, propiram, and lofexidine.

In the eighth embodiment, the invention relates to the method of the first embodiment through the seventh embodiment, wherein α-MSH activity is enhanced by administering a compound, wherein said compound triggers release of α-MSH or increases the activity of neurons that express α-MSH.

In the ninth embodiment, the invention relates to the method of the eighth embodiment, wherein said compound is a selective serotonin reuptake inhibitor (S SRI) or a specific 5-HT receptor agonist.

In the tenth embodiment, the invention relates to the method of the ninth embodiment, wherein said 5-HT receptor is selected from 5-HT1b receptor and 5-HT2c receptor.

In the eleventh embodiment, the invention relates to the method of the ninth embodiment, wherein said SSRI is selected from fluoxetine, fluvoxamine, sertraline, paroxetine, citalopram, escitalopram, sibutramine, duloxetine, and venlafaxine, and pharmaceutically acceptable salts or prodrugs thereof.

In the twelfth embodiment, the invention relates to the method of the eighth embodiment, wherein said compound is a γ-amino butyric acid (GABA) inhibitor.

In the thirteenth embodiment, the invention relates to the method of the twelfth embodiment, wherein said GABA inhibitor is a 5-HT1b receptor agonist.

In the fourteenth embodiment, the invention relates to the method of the twelfth embodiment, wherein said GABA inhibitor suppresses the expression of the AgRP gene.

In the fifteenth embodiment, the invention relates to the method of the twelfth embodiment, wherein said GABA inhibitor suppresses the production or release of AgRP.

In the sixteenth embodiment, the invention relates to the method of the ninth embodiment, wherein said 5-HT agonists inhibits the NPY/AgRP/GABA neurons.

In the seventeenth embodiment, the invention relates to the method of the twelfth embodiment, wherein said GABA inhibitor suppresses the activity of neurons that express AgRP.

In the eighteenth embodiment, the invention relates to the method of the twelfth embodiment, wherein said GABA inhibitor is topiramate.

In the nineteenth embodiment, the invention relates to the method of the eighth embodiment, wherein said compound is selected from the group consisting of a dopamine reuptake inhibitor, a norepinephrine reuptake inhibitor, a dopamine agonist, a norepinephrine releaser, a combination of a dopamine reuptake inhibitor and a norepinephrine reuptake inhibitor, and a combination of a SSRI and a norepinephrine reuptake inhibitor.

In the twentieth embodiment, the invention relates to the method of the nineteenth embodiment, wherein said compound is not phentermine.

In the twenty first embodiment, the invention relates to the method of the first embodiment, wherein said treating step comprises administering to said individual a first compound and a second compound, wherein said first compound is an opioid antagonist and said second compound enhances α-MSH activity.

In the twenty second embodiment, the invention relates to the method of the twenty first embodiment, wherein said first compound and said second compound are administered nearly simultaneously.

In the twenty third embodiment, the invention relates to the method of the twenty first embodiment, wherein said first compound is administered prior to said second compound.

In the twenty fourth embodiment, the invention relates to the method of the twenty first embodiment, wherein said first compound is administered subsequent to said second compound.

In the twenty fifth embodiment, the invention relates to a method of reducing food cravings in an individual comprising identifying an individual in need thereof and treating that individual with a combination of naltrexone and bupropion.

In the twenty sixth embodiment, the invention relates to the method of the twenty fifth embodiment, wherein the individual has a BMI greater than 30.

In the twenty seventh embodiment, the invention relates to the method of the twenty fifth embodiment, wherein the individual has a BMI greater than 25.

In the twenty eighth embodiment, the invention relates to the method of the twenty fifth embodiment, wherein the plasma concentration level of both naltrexone and bupropion follow a similar concentration profile.

In the twenty ninth embodiment, the invention relates to the method of the twenty fifth embodiment, wherein the naltrexone and the bupropion are administered substantially simultaneously.

In the thirtieth embodiment, the invention relates to the method of the twenty fifth embodiment, wherein the naltrexone is administered prior to the bupropion.

In the thirty first embodiment, the invention relates to the method of the twenty fifth embodiment, wherein the naltrexone is administered subsequent to the bupropion.

In the thirty second embodiment, the invention relates to a method of reducing cravings in an individual comprising identifying an individual in need thereof and treating that individual with a combination of naltrexone and fluoxetine.

Any of the compositions disclosed in any of the embodiments disclosed herein (including the following examples) can be used in the preparation of a medicament for the treatment of a food craving, as described herein.

### EXAMPLES

The examples below are non-limiting and are merely representative of various aspects of the invention.

### Example 1

### Reduction of Food Cravings by Administration of Bupropion and Naltrexone

This study is designed as a multicenter, randomized, double blind, and placebo-controlled Phase II clinical trial with 7 parallel groups:

### Cohort 1:

Group 1: Bupropion SR (400 mg/day) plus Naltrexone (48 mg/day)

Group 2: Bupropion SR (400 mg/day) plus Naltrexone (16 mg/day)

Group 3: Bupropion SR (400 mg/day) plus N-Placebo

Group 4: B-placebo plus Naltrexone (48 mg/day)

Group 5: B-Placebo plus N-Placebo

### Cohort 2:

Group 6: B-Placebo plus N-Placebo

Group 7: Bupropion SR (400 mg/day) plus Naltrexone (36 mg/day)

The trial consists of a screening period of 4 weeks during which patients are evaluated for eligibility, a primary treatment period of 24 weeks during which seven treatment groups are evaluated in parallel (treatment is double blind); and an extension treatment period of 24 weeks. In the extension period, groups 1, 2 and 3 continue on assigned treatment. Bupropion SR is given open label; naltrexone continues to be blinded. Groups 4 and 5 crossover to receive a combination therapy (open label bupropion SR 400 mg/day plus blinded naltrexone 36 mg/day). Both drugs are titrated as described below.

Subjects are seen at least monthly during the study. All subjects will receive ancillary therapy at baseline and at weeks 12, 24 and 36 consisting of diet instruction, advice on behavior modification and exercise. All subjects receive study drugs during the primary treatment period (24 weeks) and the extension treatment period (24 weeks). In the primary treatment period, doses of bupropion SR, B-placebo, naltrexone 12 mg, naltrexone 4 mg and N-placebo are titrated for all five groups as follows. For naltrexone 12 mg or 4 mg or N-placebo, regimen is 1 tablet in the morning for 3 days; 1 tablet in the morning and one tablet in the evening for 4 days; 2 tablets in the morning and 1 tablet in the evening for 3 weeks; and 2 tablets BID thereafter. For bupropion SR 100 mg or B-placebo, regimen is 1 tablet in the morning for 3 days; 1 tablet in the morning and one tablet in the evening for 4 days; 2 tablets in the morning and 1 tablet in the evening for the following 3 weeks; and 2 tablets BID thereafter. Morning and evening doses of Bupropion SR 100 mg of B-placebo are separated by at least 8 hours and the evening does is given as far from bedtime as possible.

In the extension treatment period, groups 1, 2, and 3 continue on assigned treatment. Groups 4 and 5 crossover to receive open label bupropion SR 400 mg/day and blinded naltrexone 32 mg/day titrated over the first 4 weeks. For naltrexone 12 mg, regimen is 1 tablet in the morning for 3 days; 1 tablet in the morning and one tablet in the evening for 4 days; and 2 tablets in the morning and 1 tablet in the evening thereafter. For bupropion SR 100 mg, regimen is 1 tablet in the morning for 3 days; 1 tablet in the morning and one tablet in the evening for 4 days; 2 tablets in the morning and 1 tablet in the evening for the following 3 weeks; and 2 tablets BID hereafter. Morning and evening doses of bupropion SR 100 mg are separated by at least 8 hours and the evening dose is given as far from bedtime as possible.

The objective of this study is to assess reduction in food craving as measured by the Food Craving Inventory (FCI). The FCI quantifies both general and specific food cravings. In the FCI, food cravings are calculated based on subjective craving ratings and the consumption of particular foods. This measure is well-accepted and validated in the art, White et al., Obesity Research, 10(2), 107-114, 2002.

The Food Craving Inventory is administered at baseline, 24 weeks and 48 weeks into the study. Efficacy for the reduction in food cravings include a reduction from baseline in the Food Craving Index (FCI) scores (total craving score and 4 subscale scores that pertain to cravings for high-fats, sweets, carbohydrates, and fast food fats) at weeks 24 and 48. A reduction in food craving is observed with either drug administered alone, or when the drugs are administered together. Combination therapies produce a synergistic effect compared to monotherapy.

### Example 2

### Reduction of Food Cravings by Administration of Fluoxetine and Naltrexone

Individuals having a BMI of greater than 25 are identified. Each individual is instructed to take one 20 mg tablet of fluoxetine (PROZAC®) on a daily basis, in addition to one 50 mg tablet of naltrexone on a daily basis.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs.

If the initial dosage is not effective at reducing food cravings to the desired extent, then the fluoxetine dosage can be increased by 20 mg per day, though never exceeding 80 mg total per day. If the initial dosage results in a more rapid weight loss than the above rate, the dosage of each of fluoxetine or naltrexone can be reduced.

Fluoxetine has a physiological half life of about 9 hours, whereas that of naltrexone is about 1.5 hours. Thus, in some cases, it is beneficial to administer one dose of fluoxetine per day in conjunction with two or three or more doses of naltrexone throughout the day. Naltrexone may also be in a time-release formulation where the dose is administered once a day, but naltrexone gradually enters the blood stream throughout the day, or in the course of a 12 hour period.

Food craving is measured using the Food Craving Inventory, at least prior to and at the completion of drug treatment. A reduction in food craving is observed after administration of fluoxetine and naltrexone.

### Example 3

### Reduction of Food Cravings by Administration of Fluoxetine and Nalmefene

Individuals having a BMI of greater than 25 are identified. Each individual is instructed to take one 20 mg tablet of fluoxetine (PROZAC®) on a daily basis. In addition, each individual is injected with 1 mL of a solution of 100 μg of nalmefene in 1 mL of saline, intravenously, intramuscularly, or subcutaneously.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs.

If the initial dosage is not effective at reducing food cravings to the desired extent, then the fluoxetine dosage can be increased by 20 mg per day, though never exceeding 80 mg total per day. In addition, the dosage of nalmefene may be increased up to 2 mL of a solution of 1 mg of nalmefene in 1 mL of saline. If the initial dosage results in a more rapid weight loss than the above rate, the dosage of each of fluoxetine or nalmefene can be reduced.

Food craving is measured using the Food Craving Inventory prior to and at the completion of drug treatment. A reduction in food craving is observed after administration of fluoxetine and nalmefene.

### Example 4

### Reduction of Food Cravings by Administration of Fluoxetine and Naloxone

Individuals having a BMI of greater than 25 are identified. Each individual is instructed to take one 20 mg tablet of fluoxetine (PROZAC®) on a daily basis. In addition, each individual is injected with 1 mL of a solution of 400 μg of naloxone in 1 mL of saline, intravenously, intramuscularly, or subcutaneously.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs.

If the initial dosage is not effective at reducing food cravings to the desired extent, then the fluoxetine dosage can be increased by 20 mg per day, though never exceeding 80 mg total per day. If the initial dosage results in a more rapid weight loss than the above rate, the dosage of each of fluoxetine or naloxone can be reduced. Food craving is measured using the Food Craving Inventory prior to and at the completion of drug treatment. A reduction in food craving is observed after administration of fluoxetine and naloxone.

### Example 5

### Reduction of Food Cravings by Administration of an Opioid Antagonist and Sibutramine

Individuals having a BMI of greater than 25 are identified. Each individual is instructed to take nalmefene, naltrexone, or naloxone in the dosage set forth in Examples 2-4. In addition, each individual is instructed to take 10 mg of sibutramine orally once a day.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs.

If the initial dosage is not effective at reducing food cravings to the desired extent, then the sibutramine dosage can be increased 15 mg per day. Dosages of sibutramine in excess of 15 mg per day are not recommended. If the initial dosage results in a more rapid weight loss than the above rate, the dosage of each of sibutramine, nalmefene, naltrexone, or naloxone can be reduced. Food craving is measured using the Food Craving Inventory prior to and at the completion of drug treatment. A reduction in food craving is observed after administration of nalmefene, naltrexone or naloxone combined with sibutramine.

### Example 6

### Reduction of Food Cravings by Administration of an Opioid Antagonist and Bupropion

Individuals having a BMI of greater than 25 are identified. Each individual is instructed to take nalmefene, naltrexone, or naloxone in the dosage set forth in Examples 2-4. In addition, each individual is instructed to take bupropion. The usual adult does is 300 mg per day, given three times daily. Dosing should begin at 200 mg per day, given as 100 mg twice daily. Based on clinical response, this dose may be increased to 300 mg per day, given as 100 mg three times daily. No single dose is to exceed 150 mg.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs. Food craving is measured using the Food Craving Inventory prior to and at the completion of drug treatment. A reduction in food craving is observed after administration of nalmefene, naltrexone or naloxone combined with bupropion.

### Example 7

### Reduction of Food Cravings by Administration of an Opioid Antagonist and Phentermine

Individuals having a BMI of greater than 25 are identified. Each individual is instructed to take nalmefene, naltrexone, or naloxone in the dosage set forth in Examples 2-4. In addition, each individual is instructed to take 37.5 mg of phentermine orally once a day.

The individuals are monitored for a period of months to determine the reduction in food cravings. The dosage may be adjusted so that each individual loses weight at a rate of 10% of initial weight every 6 months. However, the rate of weight loss for each individual may be adjusted by the treating physician based on the individual's particular needs. Food craving is measured using the Food Craving Inventory prior to and at the completion of drug treatment. A reduction in food craving is observed after administration of nalmefene, naltrexone or naloxone combined with phentermine.

### Example 8

### Reduction of Food Cravings Using Combinations with Naltrexone

In a multicenter, randomized, blinded, placebo-controlled clinical trial with 6 groups, the following drug combinations are tested:

Group 1: Fluoxetine 60 mg po QD plus Naltrexone 50 mg po QD

Group 2: Fluoxetine 60 mg po QD plus N-placebo po QD

Group 3: Bupropion-SR 150 mg po BID plus Naltrexone 50 mg po QD

Group 4: Bupropion-SR 150 mg po BID plus N-placebo po QD

Group 5: P-placebo po BID plus Naltrexone 50 mg po QD

Group 6: P-placebo po BID plus N-placebo po QD

In any of the above groups, the dosage of fluoxetine may be in the range between 6 mg and 60 mg, for example, 6 mg, 10 mg, 12 mg, 18 mg, 20 mg, 24 mg, 30 mg, 36 mg, 40 mg, 42 mg, 45 mg, 48 mg, 54 mg, and 60 mg. Bupropion may be administered in doses in the range between 30 mg and 300 mg, for example, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, and 300 mg. Naltrexone may be administered in doses in the range between 5 mg and 50 mg, for example, 5 mg, 10 mg, 15 mg, 20 mg, 25 mg, 30 mg, 35 mg, 40 mg, 45 mg, and 50 mg.

Subjects are evaluated as out-patients during this study. All subjects in this trial receive diet instruction, behavior modification advice and instruction to increase their activity, a regimen shown to give weight loss. Subjects are randomized to receive study drugs in various combinations.

Subjects in groups 5 and 6 cross-over to treatment with fluoxetine plus naltrexone or bupropion SR plus naltrexone after week 16 for the extension treatment period which provide additional data on safety of the combination therapies.

The primary endpoint is reduction in food cravings at week 16 as measured using the Food Craving Inventory prior to and at the completion of drug treatment. A reduction in food craving is observed after administration of bupropion, fluoxetine, or naltrexone. Combination therapies produce a synergistic effect compared to monotherapy.

### Example 9

### Reduction of Food Cravings by Administration of Naltrexone and Bupropion

Subjects were administered one of three treatments. Subjects in Group 1 received 400 mg/day bupropion and 16 mg/day naltrexone. Subjects in Group 2 received 400 mg/day bupropion and 48 mg/day naltrexone. Subjects in Group 3 received a double placebo treatment.

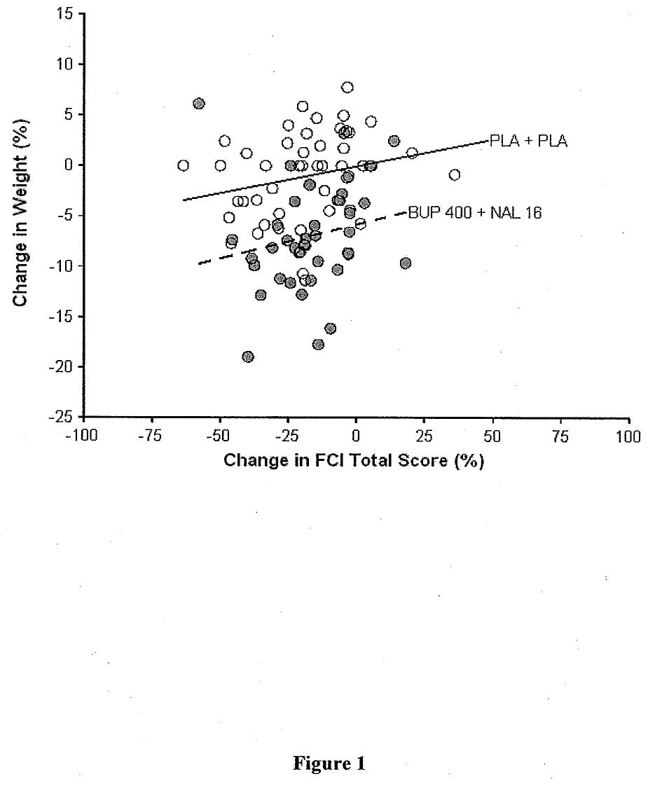

The individuals were monitored for a period of months to determine the reduction in food cravings. The body weight and the Food Craving Inventory (FCI) total score was assessed for each subject before treatment and after 24 weeks of treatment. The percentage change of both the subjects' weight and the FCI total score following treatment were calculated for both subjects in Group 1 (FIG. 1, filled symbols) and for subjects in Group 3 (FIG. 1, open symbols). In the non-placebo group, little correlation was observed between the percentage change of subjects' weight and the percentage change of the FCI total score (FIG. 1, dashed line), as compared to the correlation observed in the placebo group (FIG. 1, solid line).

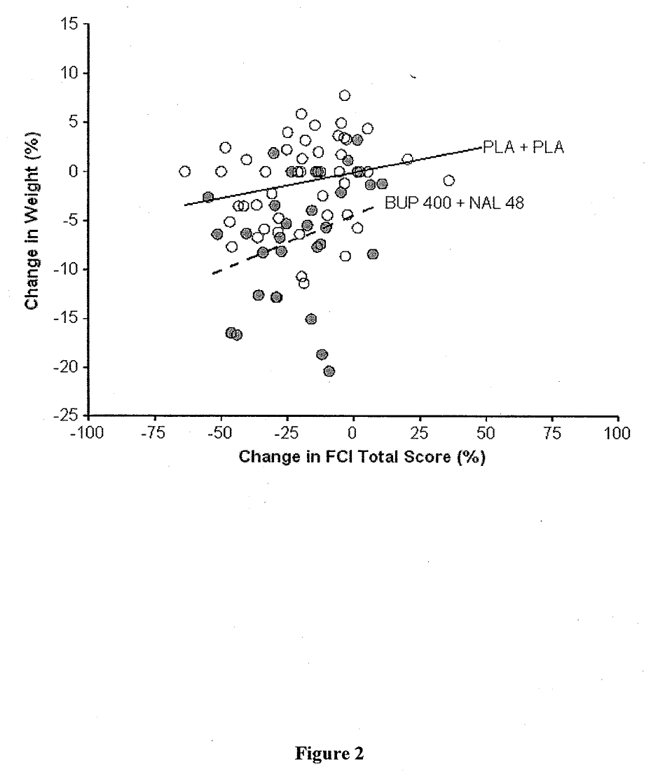

The percentage change of subjects' body weight and FCI total score was also calculated for subjects in Group 2 (FIG. 2, filled symbols) and again compared to subjects in Group 3 (FIG. 2, open symbols). In this instance, the non-placebo group showed a marked impact on FCI as increased weight loss was observed (FIG. 2, dashed line), as compared to the correlation observed in the placebo group (FIG. 2, solid line).

1-34. (canceled)

35. A method of reducing food cravings in a patient suffering therefrom comprising administering a daily dose of naltrexone or a pharmaceutically acceptable salt thereof and bupropion or a pharmaceutically acceptable salt thereof to the patient for a period of at least 4 weeks in an amount that is effective to reduce a food craving.

36. The method of claim 35, wherein the bupropion is an α-MSH agonist, triggers the release of α-MSH, and/or increases the activity of neurons that express α-MSH.

37. The method of claim 36, wherein the α-MSH activity enhancer is a selective serotonin reuptake inhibitor (S SRI).

38. The method of claim 35, wherein the naltrexone is administered to the subject prior to the bupropion.

39. The method of claim 35, wherein the naltrexone and the bupropion are combined in a single dosage form.

40. The method of claim 35, wherein the naltrexone and the bupropion are administered for a period of at least 24 weeks.

41. The method of claim 35, wherein the patient craves a food substance within a category selected from sweets, carbohydrates and fats.

42. The method of claim 41, wherein the category is sweets.

43. The method of claim 41, wherein the category is fats.

44. The method of claim 35, wherein the patient is overweight.

45. The method of claim 35, wherein the patient is obese.

46. The method of claim 35, wherein the patient has a BMI greater than 40.

47. The method of claim 35, wherein the patient has a BMI less than 25.

48. The method of claim 35, wherein the patient is pregnant.

49. The method of claim 35, wherein the patient is administered 36 mg/day of naltrexone.

50. The method of claim 35, wherein the patient is administered 400 mg/day of bupropion.

51. The method of claim 35, wherein naltrexone and bupropion are administered as part of a sustained-release formulation.
